(19) United States
(12) Patent Application Publication
(10) Pub. No.: US 2006/0035983 A1
(43) Pub. Date: Feb. 16, 2006

(54) Methods for treating fungal infections

(76) Inventors: Sheldon R. Pinnell, Durham, NC (US); Doren M. Pinnell, Durham, NC (US)

Correspondence Address: MYERS BIGEL SIBLEY & SAJOVEC, PO BOX 37428, RALEIGH, NC, 27627, US

(21) Appl. No.: 10/641,252

(22) Filed: Aug. 14, 2003

Related U.S. Application Data
(60) Provisional application No. 60/404,618, filed on Aug. 20, 2002.

(51) Int. Cl. A61K 31/08

(52) U.S. Cl. 514/723; 424/61

(57) ABSTRACT

A method of treating a mycotic infection (particularly onychomycosis) of a nail of a subject in need thereof comprises topically applying to a nail of the subject an effective antimiycotic amount of a fungicidal compound of the formula R—(O—CH₂—CH₂)ₙ—OH, wherein R is a saturated hydrocarbon or alkyl group. Compositions for carrying out the methods of the invention are also described.

### RELATED APPLICATIONS

This application claims the benefit of U.S. Provisional Patent Application Ser. No. 60/404,618, filed Aug. 20, 2002, the disclosure of which is incorporated by reference herein in its entirety.

### FIELD OF THE INVENTION

The present invention concerns topical formulations for treating fungal infections of the nails, and particularly the treatment of onychomycosis.

### BACKGROUND OF THE INVENTION

Onychomycosis is a common fungal infection of the nails that often causes substantial physical and psychological discomfort in affected individuals. Traditional treatments are by oral administration of antifungal drugs, such as fluconazole, itraconazole, and terbinafine. However, systemic treatments are costly, and can lead to harmful and undesirable side effects. Accordingly, an effective topical treatment for onychomycosis would be highly desirable.

Polyoxyethylene alkyl ethers are nonionic surfactants that have been used in topical antifungal compositions as a vehicle for other active antifungal agents. For example, U.S. Pat. No. 6,143,794 to Chaudhuri et al. proposes a composition for treating nail fungal disease containing a benzylamine compound as the active antifungal agent. The composition optionally contains a surfactant present in an amount of 0% to 10% by weight to aid in the penetration of the antigfungal agent through the nailplate. Representative nonionic surfactants include polysorbates, polyoxyethylene 4 laurly ether, and the like.

U.S. Pat. No. 6,319,509 to Richter et al. proposes a topical antifungal formulation containing the allylamine compound terbinafine as the active anti-mycotic agent. The formulation optionally includes a surfactant, such as a polyethylene glycol alkyl ether, in an amount of approximately 2% by weight to help solubilize the drug, especially in vehicles containing water.

U.S. Pat. No. 5.827,870 to Chodosh et al. proposes an antimicrobial composition useful for the topical treatment of microbial infections. The composition preferably contain a quaternary ammonium compound as an antimicrobial agent, and a keratolytic agent in the amount of from about 0.05-5% by weight to increase the effectiveness of the antimicrobial agent. Keratolytic agents useful in the composition include allantoin, triacetin, acetic acid, salicylic acid, and polyoxyethylene lauryl ether.

U.S. Pat. No. 4,775,678 to Su et al. proposes a topical cream or lotion formulation containing the imidazole-derivative clotrimazole as the antifungal compound. Formulations of the invention include a non-ionic surfactant in the amount of approximately 2.25% by weight, which forms an oil-in-water emulsion cream base. Examples of surfactants include ceteth-20, steareth-2, steareth-20 or mixtures thereof, and the like.

U.S. Pat. No. 5.461,068 to Thaler et al. proposes a stable solvent system for antifungal imidazole derivatives. The solvent system contains a non-ionic or amphoteric surfactant, such as Brij 30 or Brij 96, in an amount of 0 to 5% by weight. Many topical antifungal agents used to treat onychomycosis are known to illicit contact allergies in some patients, including imidazole derivatives (see e.g., Cont. Derm., 33(4), 282 (1995)), quaternary ammonium compounds (see e.g., Cont. Derm., 1(5), 316 (1975)), and terbanifine (see e.g., Pediatr. Infect. Dis. J., 16(6), 545 (1997)). It would thus be beneficial to have alternative formulations available.

### SUMMARY OF THE INVENTION

The present invention relates to methods of topically treating onychomycosis via compositions containing polyoxyethylene alkyl ethers, which are widely used in cosmetic preparations, as the active ingredient.

A first aspect of the present invention is a method of treating a mycotic infection (particularly onychomycosis) of a nail of a subject in need thereof, comprising topically applying to a nail of the subject an effective antimycotic amount of a fungicidal compound of the formula R—(O—CH₂—CH₂)ₙ—OH, wherein R is a saturated hydrocarbon or alkyl group, and is preferably a straight-chain saturated hydrocarbon (e.g., of from 4, 6 or 8 to 24, 26 or 28 carbons).

Stated otherwise, the present invention provides a method of treating a mycotic infection (particularly onychomycosis) of the nails of a subject in need thereof, comprising topically applying to the nails of the subject an antifungal composition, the antifungal composition comprising, consisting of, or consisting essentially of: a fungicidal compound of the formula R—(O—CH2-CH2)ₙ-OH as described above in combination with a pharmaceutically acceptable topical carrier. The composition is preferably free of or devoid of other antimycotic compounds, such as imidazole derivative compounds, quaternary ammonium compounds, allylamine compounds, and benzylamine compounds.

The foregoing and other objects and aspects of the present invention are explained in greater detail in the specification set forth below.

### DETAILED DESCRIPTION OF THE PREFERRED EMBODIMENTS

The invention will now be described with respect to preferred embodiments described herein. It should be appreciated however that these embodiments are for the purpose of illustrating the invention, and are not to be construed as limiting the scope of the invention as defined by the claims.

“Nail” as used herein refers to any type of nail, including finger nails and toe nails. Toe nails are particularly preferred. While “nail” is referred to singly herein, it will be appreciated that treatment of one nail will include one or more nails, any will encompass treatment of a plurality of nails. The term “nail” is intended to be inclusive of “hoof” unless otherwise specifically excluded.

The term “treat” as used herein refers to any type of treatment that imparts a benefit to a patient afflicted with a disease, including improvement in the condition of the patient (e.g., in one or more symptoms), delay in the progression of the disease, etc.

The term “pharmaceutically acceptable” as used herein means that the compound or composition is suitable for administration to a subject to achieve the treatments described herein, without unduly deleterious side effects in light of the severity of the disease and necessity of the treatment.

Active compounds of the present invention may optionally be administered in conjunction with other compounds useful in the treatment of onychomycosis or other fungal infections. The other compounds may optionally be administered concurrently. As used herein, the word “concurrently” means sufficiently close in time to produce a combined effect (that is, concurrently may be simultaneously, or it may be two or more events occurring within a short time period before or after each other).

As used herein, the administration of two or more compounds “in combination” means that the two compounds are administered closely enough in time that the presence of one alters the biological effects of the other. The two compounds may be administered simultaneously (i.e., concurrently) or sequentially. Simultaneous administration may be carried out by mixing the compounds prior to administration, or by administering the compounds at the same point in time but at different anatomic sites or using different routes of administration.

The phrases “concurrent administration,” “administration in combination,” “simultaneous administration” or “administered simultaneously” as used herein, interchangeably mean that the compounds are administered at the same point in time or immediately following one another. In the latter case, the two compounds are administered at times sufficiently close that the results observed are indistinguishable from those achieved when the compounds are administered at the same point in time.

Human subjects may be male or female and may be of any suitable age, including infants, children, adolescents and adults.

The present invention is primarily concerned with the treatment of human subjects, but the invention may also be carried out on animal subjects, particularly mammalian subjects such as mice, rats, dogs, cats, livestock and horses for veterinary purposes, and for drug screening and drug development purposes.

Examples of fungal infections in the hooves of horses that may be treated by the methods and compositions of the present invention include, but are not limited to, thrush, hoof wall fungus, and white line disease. Since hoof wall fungus and white line disease are caused by onychomycosis, they are particularly preferred.

While the methods of the present invention are primarily concerned with treating fungal infection of the nails, the present invention may also be used to treat fungal infections of the skin and/or hair, such as ringworm and animal ringworm. Such methods may be carried out by topically applying the compositions described herein to an infected area of the skin and/or hair, in like manner and dose as described herein with respect to nails,

In addition, the compositions described herein may be used to treat or combat fungal infection of substrates from which fungus may be spread to a human or animal, such as ground, pens, bedding, etc., by topically applying the compositions described herein to such substrates in like manner and concentration as described herein, to combat/slow the growth of, kill, and/or sterilize, etc., the fungus in areas from which infection might otherwise spread to a human or animal host.

1. Active Compounds.

The methods of the present invention include the administration of compounds of Formula I, while pharmaceutical compositions of the present invention comprise compounds of Formula I. As used herein, a compound of Formula I is as follows:
R—(O—CH2-CH2)ₙ-OH (I)
wherein R is a saturated hydrocarbon, preferably C4, C6, C8 or C12 to C18, C24, C26 or C28 alkyl, and n is 1, 2, 4 or 6 to 16, 18 or 24 (e.g., 2, 3, 4, 5, 6, 7, 8, 9, 10, 11, 12, 13, 14, 15, 16, 17, 18, 19, 20, 21, 22, 23, 24).

Compounds illustrative of the compounds of Formula (I) above include:

Polyoxyethylene 4 lauryl ether, marketed under the name “Brij® 30” (e.g., Sigma-Aldrich product no. 23,598-9), wherein R is 12 and n is 4, giving a structure of: CH₃(CH₂)₁₁(OCH₂CH₂)₄OH.

Polyoxyethylene 23 lauryl ether, marketed under the name “Brij® 35” (e.g., Sigma-Aldrich product no. 85,836-6), wherein R is 12 and n is 23, giving a structure of: CH₃(CH₂)₁₁(OCH₂CH₂)₂₃OH.

Polyoxyethylene 2 cetyl ether, marketed under the name “Brij® 52” (e.g., Sigma-Aldrich product no. 38,883-1) 23,599-7, wherein R is 16 and n is 2, giving a structure of: CH₃(CH₂)₁₅(OCH₂CH₂)₂OH

Polyoxyethylene 10 cetyl ether, marketed under the name “Brij® 56” (e.g., Sigma-Aldrich product no. 38,885-8), wherein R is 16 and n is 10, giving a structure of: CH₃(CH₂)₁₅(OCH₂CH₂)₁₀OH.

Polyoxyethylene 20 cetyl ether, marketed under the name “Brij® 58” (e.g., Sigma-Aldrich product no. 23,599-7), wherein R is 16 and n is 20, giving a structure of: CH₃(CH₂)₁₅(OCH₂CH₂)₂₀OH.

Polyoxyethylene 2 stearyl ether, marketed under the name “Brij® 72” (e.g., Sigma-Aldrich product no. 38,888-2), wherein R is 18 and n is 2, giving a structure of: CH₃(CH₂)₁₇(OCH₂CH₂)₂OH.

Polyoxyethylene 10 stearyl ether, marketed under the name “Brij® 76” (e.g., Sigma-Aldrich product no. 38,889-0), wherein R is 18 and n is 10, giving a structure of: CH₃(CH₂)₁₇(OCH₂CH₂)₁₀OH.

Polyoxyethylene 20 stearyl ether, marketed under the name “Brij® 78” (e.g., Sigma-Aldrich product no. 23,600-4), wherein R is 18 and n is 20 giving a structure Of: CH₃(CH2)₁₇(OCH₂CH₂)₂₀OH.

Additional examples of compounds that may be used to carry out the present invention will be apparent to those skilled in the art based upon the information set forth above.

2. Pharmaceutical Formulations.

The active compounds described above may be formulated for administration in a pharmaceutical carrier in accordance with known techniques. See, e.g., Remington, The Science And Practice of Pharmacy (9th Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. One or more active compounds may be incorporated in the formulations of the invention, which may be prepared by any of the well known techniques of pharmacy consisting essentially of admixing the components, optionally including one or more accessory ingredients.

Formulations suitable for topical application to the nails preferably take the form of a solution, liquid, ointment, cream, lotion, paste, gel, spray, aerosol, and/or oil. Acceptable carriers for topical application to the nails which may be used include petroleum jelly, water, lanoline, polyethylene glycols, alcohols, transdermal enhancers, and combinations thereof.

In addition to compounds of formula (I) or their salts, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include, but are not limited to, methylparaben, propylparaben, and benzyl alcohol. Where compositions of the invention are described as being devoid or free of other antimycotic agents, it will be understood that this referrs to other agents that treat the nail of the subject, and not agents that prevent microbial growth within the composition itself. The microbial preservative is typically employed when the formulation is placed in a vial designed for multidose use.

In one embodiment, a composition useful for treating a mycotic infection in the nail of a subject in need thereof, comprises, consists of, or consists essentially of:

(a) an effective antimycotic amount of a fungicidal compound as described above (typically included in an amount of from about 0.1, 0.5 or 1 to 5, 10 or 15 percent by weight);

(b) a nail moisturizer such as hyaluronic acid, alpha hydroxy acids, petroleum jelly, ceramide, lanolin, etc. (typically included in an amount of from about 0.1, 0.5 or 1 to 2, 3 or 5 percent by weight);

(c) water (to balance); and

(d) optionally a nail hardener such as biotin or zinc. Inclusion of the nail moisturizer advantageously serves to prevent drying of the nail by the topical application of the surfactant. The composition may be provided in any suitable form, such as a liquid, cream or gel.

3. Dosage and Route of Administration.

As noted above, the present invention provides pharmaceutical formulations comprising the active compounds (including the pharmaceutically acceptable salts thereof), in pharmaceutically acceptable carriers for topical, or transdermal administration.

The therapeutically effective dosage of any one active agent, the use of which is in the scope of present invention, will vary somewhat from compound to compound, and patient to patient, and will depend upon factors such as the age and condition of the patient and the route of delivery. Such dosages can be determined in accordance with routine pharmacological procedures known to those skilled in the art.

In one embodiment, the active antifungal compositions described herein are included in the formulations for topical delivery in an amount of at least 5, 8 or 10 percent by weight.

The duration of the treatment may be once per day for a period of at least two to three weeks or until the condition is essentially controlled. In one embodiment, the duration is one dose per day until the affected nail or nails grow out, which may require up to two years. Lower doses given less frequently can be used prophylactically to prevent or reduce the incidence of recurrence of the infection.

The foregoing is illustrative of the present invention, and is not to be construed as limiting thereof. The invention is defined by the following claims, with equivalents of the claims to be included therein.

### EXAMPLE 1

### Antifungal Susceptibility Testing

A number of polyoxyethylene alkyl ethers were tested for fungicidal activity against Trichophyton rubrum cultures in vitro according to standard methods (see, Approved Standard M27-A, National Commitee for Clincal Laboratory Standards, 1997). Briefly, inocula from Trichophyton rubrum were harvested from agar cultures, suspended in 0.85% saline, and diluted to a verified final concentration of 103 colony-forming units (CFU) per ml. Suspensions were then treated with various polyoxyethylene alkyl ethers at a range of concentrations, and incubated for 7 days at 30° C. Minimum inhibitory concentrations (MICs) were determined as the lowest concentration of compound that inhibited 100% of fungal growth, as compared with a polyoxyethylene alkyl ether-free control suspension. Minimum fungicidal concentrations (MFCs) were determined as the lowest concentration of compound that killed at least 97% of the original inoculum, as compared with the verified inoculum count. Data are shown in Table 1.

These results show that the chemical specificity of the surfactant is important for the fungicidal effect. The fungicidal activity observed for these surfactants is not a general detergent effect. Moreover, it is not a general effect of nonionic surfactants or of critical micelle content (CMC). The fungicidal effect was most sensitive with polyoxyethylene alkyl ethers. Adding a double bond to the alkyl side chain resulted in loss of the effect (See Oleth-2 and Oleth-20 compared to Ceteth-2 and Ceteth-20.

TABLE 1

| | TRIVIAL | | | | MIC80 | MIC100 | MFC |
|---|---|---|---|---|---|---|---|
| COMPOUND | NAME | CHEMICAL NAME | TYPE | CMC (mM) | (μg/ml) | (μg/ml) | (μg/ml) |
| CHAPS | | | Zwitterionic | 6 to 10 | 500 | 1000 | |
| Zwittergent | | | Zwitterionic | 0.1-0.4 | 15.69 | 31.25 | |
| Tween 20 | | Polyoxyethylenesorbitan monolaurate | Nonionic | 0.059 | 125 | 500 | 1000 |
| Tween 60 | | Polyoxyethylenesorbitan monopalmitate | Nonionic | 0.059 | 250 | 500 | 1000 |
| Tween 80 | | Polyoxyethylenesorbitan monooleate | Nonionic | 0.059 | 250 | 1000 | 1000 |
| n-octyl-β-D- | | | Nonionic | 20-25 | 31.25 | 1000 | |
| glucopyranoside | | | | | | | |
| n-octyl-β-D- | | | Nonionic | 9 | 500 | 1000 | |
| thioglucopyranoside | | | | | | | |
| Triton X-100 | | Polyethylene glycol tert-octylphenyl ether | Nonionic | 0.2-0.9 | 31.25 | 1000 | |
| BRIJ 30 | Laureth-4 | Polyoxyethylene 4 lauryl ether | Nonionic | | 0.98 | 1.95 | 1.95 |
| BRIJ 35 | Laureth-23 | Polyoxyethylene 23 lauryl ether | Nonionic | 0.09 | 0.49 | 0.98 | 1.95 |
| BRIJ 52 | Ceteth-2 | Polyoxyethylene 2 cetyl ether | Nonionic | | 0.98 | 3.9 | 7.8 |
| BRIJ 58 | Ceteth-20 | Polyoxyethylene 20 cetyl ether | Nonionic | | 0.49 | 0.98 | 1.95 |
| BRIJ 68 | Cetearth-20 | Polyoxyethylene 20 cetyl/stearyl ether | Nonionic | | 1.95 | 3.9 | 15.6 |
| BRIJ 72 | Steareth-2 | Polyoxyethylene 2 stearyl ether | Nonionic | | 0.98 | 0.98 | 3.9 |
| BRIJ 78 | Steareth-20 | Polyoxyethylene 20 stearyl ether | Nonionic | | 0.98 | 1.95 | 3.9 |
| BRIJ 93 | Oleth-2 | Polyoxyethylene 2 oleyl ether | Nonionic | | 250 | 250 | 1000 |
| BRIJ 98 | Oleth-20 | Polyoxyethylene 20 oleyl ether | Nonionic | | 250 | 500 | 1000 |

### EXAMPLE 2

### Additional Fungal Species Susceptibility Testing

Trichophyton rubrum is the causative organism in 68-100% of patients in onychomycosis trials (Crawford et al. (2002) Archives Dermatol. 138:811-816). Trichophyton Mentagrophytes is the causative organism in most of the rest. Although yeast and other nondermatophytes can cause onychomycosis, the incidence is small. Additional fungal species were tested for susceptibility to treatment with Laureth-4 as described in Example 1. These data are shown in Table 2 below. These results indicate that non-Trichophyton species are not particularly susceptible to Laureth-4 and that the fungicidal activity of Laureth-4 on Trichophyton rubrum is not a general effect of surfactant on fungal organisms.

TABLE 2

| | TRIVIAL | CHEMICAL | | | MIC80 | MIC100 | MFC |
|---|---|---|---|---|---|---|---|
| COMPOUND | NAME | NAME | TYPE | ORGANISM | (μg/ml) | (μg/ml) | (μg/ml) |
| BRIJ 30 | Laureth-4 | Polyoxyethylene 4 | Nonionic | Trichophyton | 0.98 | 1.95 | 1.95 |
| | | lauryl ether | | Rubrum | | | |
| BRIJ 30 | Laureth-4 | Polyoxyethylene 4 | Nonionic | Trichophyton | 5 | 625 | 625 |
| | | lauryl ether | | mentagrophytes | | | |
| BRIJ 30 | Laureth-4 | Polyoxyethylene 4 | Nonionic | Candida | >5000 | >5000 | >5000 |
| | | lauryl ether | | Albicans | | | |
| BRIJ 30 | Laureth-4 | Polyoxyethylene 4 | Nonionic | Candida | 15.5 | >5000 | >5000 |
| | | lauryl ether | | Parapsilosis | | | |
| BRIJ 30 | Laureth-4 | Polyoxyethylene 4 | Nonionic | Scopulariopsis | 78 | >5000 | >5000 |
| | | lauryl ether | | brevicaulis | | | |
| BRIJ 30 | Laureth-4 | Polyoxyethylene 4 | Nonionic | Aspergillus | 39 | >5000 | >5000 |
| | | lauryl ether | | flavus | | | |
| BRIJ 30 | Laureth-4 | Polyoxyethylene 4 | Nonionic | Fusarium solani | 78 | >5000 | >5000 |
| | | lauryl ether | | | | | |

The foregoing is illustrative of the present invention, and is not to be construed as limiting thereof. The invention is defined by the following claims, with equivalents of the claims to be included therein.

That which is claimed is:

1. A method of treating a mycotic infection of a nail of a subject in need thereof, comprising topically applying to a nail of said subject an effective antimycotic amount of a fungicidal compound of the formula
R—(O—CH₂—CH₂)ₙ—OH
wherein
R is a C12-C18 alkyl and n is 1 to 24.

2. The method of claim 1 wherein R is CH₃(CH₂)₁₁ and n is 4.

3. The method of claim 1 wherein R is CH₃(CH₂)₁₁ and n is 23.

4. The method of claim 1 wherein R is CH₃(CH₂)₁₅ and n is 20.

5. The method of claim 1 wherein R is CH₃(CH₂)₁₇ and n is 2.

6. The method of claim 1 wherein R is CH₃(CH₂)₁₇, and n is 20.

7. The method of claim 1, wherein R is a straight-chain saturated hydrocarbon.

8. The method of claim 1, wherein said mycotic infection is onychomycosis.

9. The method of claim 1, wherein said subject is a human subject.

10. The method of claim 1, wherein said nail is selected from the group consisting of finger nails and toe nails.

11. A method of treating a mycotic infection of the nails of a subject in need thereof, comprising topically applying to the nails of said subject an antifungal composition consisting essentially of:
a fungicidal compound of the formula R—(O—CH2-CH12)ₙ-OH, wherein R is C12-C18 alkyl and n is 1 to 24, in combination with a pharmaceutically acceptable topical carrier.

12. The method of claim 11 wherein R is CH₃(CH₂)₁₁ and n is 4.

13. The method of claim 11 wherein R is CH₃(CH₂)₁₁ and n is 23.

14. The method of claim 11 wherein R is CH₃(CH₂)₁₅ and n is 20.

15. The method of claim 11 wherein R is CH₃(CH₂)₁₇ and n is 2.

16. The method of claim 11 wherein R is CH₃(CH₂)₁₇, and n is 20.

17. The method of claim 11, wherein R is a straight-chain saturated hydrocarbon.

18. The method of claim 11, wherein said mycotic infection is onychomycosis.

19. The method of claim 11, wherein said subject is a human subject.

20. The method of claim 11, wherein said nail is selected from the group consisting of finger nails and toe nails.

21. A method of treating a mycotic infection of the nails of a subject in need thereof, comprising topically applying to the nails of said subject an effective antimycotic amount of a composition, said composition comprising a fungicidal compound of the formula:
R—(O—CH₂—CH₂),—OH
wherein R is a C12-C18 alkyl and n is 1 to 24; and
said composition is devoid of other antimycotic compounds.

22. The method according to claim 21, wherein said composition is devoid of other antimycotic compound selected from the group consisting imidazole derivative compounds, quaternary ammonium compounds, allylamine compounds, and benzylamine compounds.

23. A composition useful for treating a mycotic infection in the nail of a subject in need thereof, said composition comprising:
(a) an effective antimycotic amount of a fungicidal compound of the formula
R—(O—CH₂—CH₂)ₙ—OH
wherein R is C12-C18 alkyl and n is 1 to 24;
(b) a nail moisturizer; and
(c) water.

24. A composition according to claim 23, wherein said nail moisturizer is selected from the group consisting of hyaluronic acid, alpha hydroxy acids, petroleum jelly, ceramide, and lanolin.

25. A composition according to claim 23, further comprising:
(d) a nail hardener.

26. A composition according to claim 25, wherein said nail hardener is selected from the group consisting of biotin and zinc.
